(19) United States
(12) Patent Application Publication
(10) Pub. No.: US 2026/0364240 A1
(43) Pub. Date: Nov. 19, 2026

(54) METHOD AND DEVICE FOR ADAPTIVE DETECTION OF GAS BUBBLES IN AN INFUSION TUBE OR BLOOD TUBE USING ULTRASOUND

(71) Applicant: B. Braun Melsungen AG, Melsungen (DE)

(72) Inventors: Johannes Krapf, Bebra (DE); Rolf Hollstein, Alheim-Heinebach (DE); Ernst Bressau, Malsfeld (DE)

(21) Appl. No.: 19/678,706

(22) Filed: May 15, 2026

(30) Foreign Application Priority Data
May 16, 2025 (EP) 25177075.6

(51) Int. Cl. A61M 1/36

(52) U.S. Cl. CPC A61M 1/3626; A61M 2205/3375

(57) ABSTRACT

Detection and quantification of gas bubbles in an infusion tube or blood tube is performed with an ultrasound-based gas sensor. Ultrasonic pulses are applied to the infusion tube or blood tube using an ultrasonic transmitter. The pulses are detected by an ultrasonic receiver. A sensor signal output is evaluated by a control and evaluation unit. The sensor signal or a signal derived from the sensor signal is compared with a predetermined threshold value to detect gas bubbles. An alarm is output when the sensor signal exceeds the threshold value. During evaluation of the sensor signal, a parameter is automatically adjusted or varied. The parameter can be a transmission energy introduced into the infusion tube or blood tube by the pulses and/or the threshold value for triggering the alarm signal.

### CROSS-REFERENCE TO RELATED APPLICATIONS

This application claims priority under 35 U.S.C. § 119 to European Application No. 25177075.6, filed on May 16, 2025, the content of which is incorporated by reference herein in its entirety.

### FIELD

The present invention relates to the technical field of medical device technology, in particular systems for monitoring fluid flows in infusion tubing of infusion devices or in blood tubing of dialysis devices. The invention relates to a method and a device for detecting and, if necessary, quantifying gas bubbles, in particular air bubbles, within an infusion tubing through which an infusion medium flows or a blood tubing through which blood flows, using an ultrasonic sensor system. The invention is particularly suitable for use in infusion and dialysis systems as well as extracorporeal blood circuits.

### BACKGROUND

In the prior art, the detection of air or other gases in fluid tubing, particularly in infusion tubing, is frequently implemented using ultrasonic sensors. In this process, ultrasonic pulses are sent through the tubing, and the change in signal attenuation or signal scattering caused by gas bubbles contained in the medium is evaluated. A comparison with a fixed threshold determines the presence of gas inclusions, particularly air bubbles.

However, particularly at very low flow rates, such as those frequently encountered in infusion systems, small gas bubbles can become trapped in the sensor area or slowly aggregate into larger bubbles. This behavior depends heavily on the tube’s position, tube material, and sensor geometry. Additionally, the infusion tube and the infusion solution within it are often subject to physical fluctuations, such as temperature, which influence the sensor signal. Under these conditions, the fixed thresholds of the prior art lead to undesirable system responses.

### SUMMARY

It is therefore the objective of the present invention to provide a method and a device that enable reliable detection and, if necessary, also quantification of gas bubbles in an infusion tube or blood tube, even under conditions of low flow rates or when gas bubbles are temporarily trapped in the sensor area. The objective is, in particular, to reduce the dependence of gas detection on the dynamic position of individual gas bubbles.

Accordingly, an ultrasound-based method for detecting gas bubbles in an infusion tube or blood tube is provided. In this method, a sensor signal generated by an ultrasound receiver is evaluated in a control and evaluation unit and compared with a threshold value. An alarm signal is emitted if the sensor signal exceeds or falls below the threshold value. It is essential that, during the evaluation, at least one of the following parameters is automatically adjusted or varied: (i) the transmission energy of the ultrasonic pulses introduced into the tube and/or (ii) the threshold value for triggering the alarm.

The control of these parameters allows for dynamic adaptation to changing physical conditions such as medium type, temperature, hose material, flow behavior, and air distribution. This enables stable detection even under challenging conditions. In particular, the method is capable of tolerating so-called “stationary air bubbles” – that is, gas bubbles that remain in the sensor area for extended periods – and correctly accounting for them as they are transported further.

Furthermore, a device is provided that comprises, among other things, an ultrasonic transmitter, an ultrasonic receiver, a control and evaluation unit, a comparison unit, and an alarm unit. A control or regulation unit is additionally provided to automatically adjust at least one of the above-mentioned parameters – transmission power or alarm threshold. The device is thus technically configured to carry out the method according to the invention.

The advantages and features of the described embodiments of the method can be applied in a corresponding manner to the device as well. Conversely, the technical embodiments of the device serve, in particular, to implement and optimize the method. To the extent that features are described as method-specific, they therefore apply mutatis mutandis to the corresponding device and vice versa.

In a preferred embodiment, the transmission energy is adjusted by controlling the number of successive ultrasonic pulses and/or by regulating the energy content of individual pulses. This measure allows for particularly flexible adjustment of the energy input to different tube and fluid properties.

In a preferred embodiment, the energy content of individual ultrasonic pulses can be influenced by controlling the charging time of an inductor arranged in the transmission circuit of the ultrasonic transmitter. By varying the charging time, the energy stored in the inductor is regulated, which is subsequently used during discharge to drive the ultrasonic transmitter. The longer the charging time (at a constant supply voltage), the greater the energy stored in the inductor’s magnetic field, and the higher the energy content of the generated ultrasonic pulse. This allows for precise control of the coupled ultrasonic energy depending on external conditions such as hose material, filling medium, or gas content. The use of a controllable charging time to influence the energy content represents a particularly simple and robust method for dynamic power adjustment during operation, without the need for complex analog power control.

Advantageously, the threshold value for triggering the alarm signal and/or the transmission energy is dynamically adjusted depending on the sensor signal’s progression over time.

In a preferred embodiment, the alarm threshold and/or transmission power are controlled based on a predetermined target range for the sensor signal within which gas bubbles and liquid can be reliably distinguished. By comparing current measured values with the target range, automatic readjustment of the transmission power can occur, leading to more stable sensor operation.

Advantageously, the automated adjustment is controlled by an algorithm implemented in the control and evaluation unit, which analyzes characteristic signal waveforms and assigns them to different physical situations, in particular stationary or moving gas bubbles.

A particularly advantageous version of the method involves calculating averages or other statistical measures of the sensor signal over a specified time interval; if the maximum difference among the measured values falls below or exceeds a specified threshold, the corresponding measured values are discarded, and a new averaging process is initiated.

In a preferred embodiment, the algorithm is based on a lookup table or an adaptive model that has been trained in advance or during operation. This allows for adaptive adjustment to specific device configurations or patient conditions.

Advantageously, in addition to detection, a calculation of the gas volume is also performed. For this purpose, the departure of gas bubbles from the sensor area is detected and quantified based on signal trends and changes. This can be achieved in particular by performing a before-and-after comparison when there is a sudden increase in the sensor signal due to a gas bubble leaving the sensor area, in order to detect the disappearance of the bubble and to approximately determine its volume.

In a preferred embodiment, an alarm is triggered only if the threshold value is exceeded for a defined minimum duration or if the slope of the sensor signal exceeds a certain limit. This measure serves to prevent unwanted alarms in the event of brief signal deviations.

In a preferred embodiment, a plausibility check is performed to distinguish genuine gas bubbles from signal artifacts, such as those caused by environmental influences.

Advantageously, the evaluation is performed depending on the operating mode of the infusion device. For example, different evaluation algorithms may be used in preparation mode than in therapy mode.

In a preferred embodiment, the control system is additionally fed by further input information, such as the type of infusion medium, the current flow rate, or the geometric arrangement of the tubing within the sensor. This data can be incorporated into the control or regulation strategy to further increase detection accuracy.

In a preferred embodiment, the control and evaluation unit may be fully integrated into the gas sensor. This results in a particularly compact and space-saving design in which all signal processing steps, including threshold comparison, regulation of transmission power, and alarm triggering, take place within the sensor housing. Alternatively, the control and evaluation unit may also be implemented partially or completely as a separate component , for example in the form of an external control device or as part of a higher-level infusion or dialysis system and/or as a cloud service. Such offloading can be particularly advantageous when more complex signal processing or communication with other system components is required.

The gas sensor may be configured as a transmission or reflection system. In a transmission configuration, the ultrasonic transmitter and receiver are arranged on opposite sides of the infusion tube so that the ultrasonic pulses pass completely through the tube. This arrangement enables direct detection of signal attenuation caused by gas bubbles within the tube. Alternatively, the gas sensor may be designed as a reflection system in which the transmitter and receiver are arranged on the same side of the tube in the form of an ultrasonic transmitter and receiver unit, and an ultrasonic reflector is mounted on the opposite side. In this configuration, the emitted ultrasonic signal is reflected back by the ultrasonic reflector and detected by the receiver. The reflection arrangement allows for a particularly compact design and can be advantageous, for example, when access is possible to only one side of the tube. Both variants are equally suitable for carrying out the method according to the invention. Additionally, the measurement accuracy of the method is improved by doubling the distance traveled by the emitted ultrasonic signal.

The features, advantages, and areas of application of the invention described thus far in connection with infusion systems apply equally to blood circuit systems of dialysis machines. From a technical and functional standpoint, the blood tubing of a dialysis machine is subject to requirements regarding air bubble monitoring that are comparable to those of an infusion tubing.

In hemodialysis procedures in particular, a safe and gas-bubble-free connection between the extracorporeal circuit and the patient is of central importance. A device such as that described in the present invention can also ensure increased operational safety here by enabling reliable detection and, if necessary, quantification of gas bubbles or an automatic alarm in the event of deviations. The principles of the invention can therefore be directly applied to the blood tubing of a dialysis machine without requiring significant technical modifications. The scope of protection of this application thus expressly extends to applications in the field of dialysis technology.

In this application, the term “tube” may preferably refer to a flexible tube, but alternatively may also refer to a comparatively rigid line, a pipe, or a flow channel, provided that the described ultrasonic measurement method is compatible with it.

In summary, the invention relates to a method and a device for the ultrasound-based detection and optional quantification of gas bubbles in an infusion tube or blood tube. The automated control or regulation of the transmission power and/or the alarm threshold based on sensor signals enables robust, fault-tolerant, and adaptively adjustable air detection. The invention is particularly suitable for use in medical infusion systems with low flow rates, in which conventional detection systems frequently lead to unwanted alarms or system reactions.

### BRIEF DESCRIPTION OF THE DRAWINGS

FIG. 1 shows a cross-sectional view of an infusion tube with an associated gas sensor, implemented as a transmission ultrasound path with an opposing transmitter and receiver.

FIG. 2 shows a variation of the arrangement shown in FIG. 1, comprising a reflected ultrasonic path with a reflector and a combined transmitter-receiver element on one side of the infusion tubing.

FIG. 3 shows a block diagram of a system for detecting gas bubbles in an infusion tube, based on an arrangement according to FIGS. 1 or 2, with a number of components for regulating the transmission power by varying the charging time and/or the number of transmission pulses.

FIG. 4 shows a block diagram of a corresponding system with components for adjusting the alarm threshold as a function of the sensor signal.

FIG. 5 shows the temporal progression of a damping signal detected by a gas sensor in the sensor area, with examples of different accumulation and detachment processes of air bubbles.

### DETAILED DESCRIPTION

FIG. 1 shows a preferred embodiment of the gas sensor 1 according to the invention and its arrangement around the infusion tube 4, in which gas bubbles are to be detected. This is a transmission path in which an ultrasonic transmitter 2 and an ultrasonic receiver 3 are arranged on both sides opposite the infusion tube 4. The infusion tube 4 is enclosed by the ultrasonic transmitter 2 and the ultrasonic receiver 3. The section of the infusion tube 4 detected by the gas sensor 1 is the sensor region 5 between the ultrasonic transmitter 2 and the ultrasonic receiver 3. This arrangement allows the infusion tube 4, and in particular the sensor region 5, to be fixed in place, thereby minimizing measurement errors. At the same time, the opposing positioning of the ultrasonic transmitter 2 and the ultrasonic receiver 3 ensures that the entire cross-section of the infusion tube 4 is detected during the measurement.

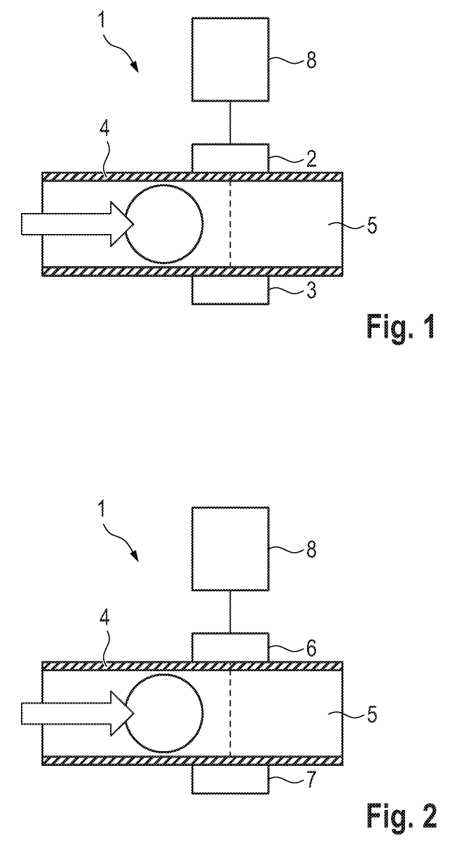

Another preferred and inventive embodiment of the gas sensor 1 is shown in FIG. 2. In this embodiment, the ultrasonic transmitter 2 and the ultrasonic receiver 3 are combined in an ultrasonic transmitter and receiver unit 6 in the form of a single component. This has the particular advantage of reducing the number of electrical components and enabling a more compact design. In this embodiment, the infusion tube 4 is enclosed by the ultrasonic transmitter and receiver unit 6 and additionally by an ultrasonic reflector 7, which is positioned opposite the ultrasonic transmitter and receiver unit. This variant also has the advantage that the position of the infusion tube 4 and the sensor region 5 is ensured. Additionally, in this embodiment, the ultrasonic pulses pass through the sensor region 5 a second time, thereby increasing measurement accuracy.

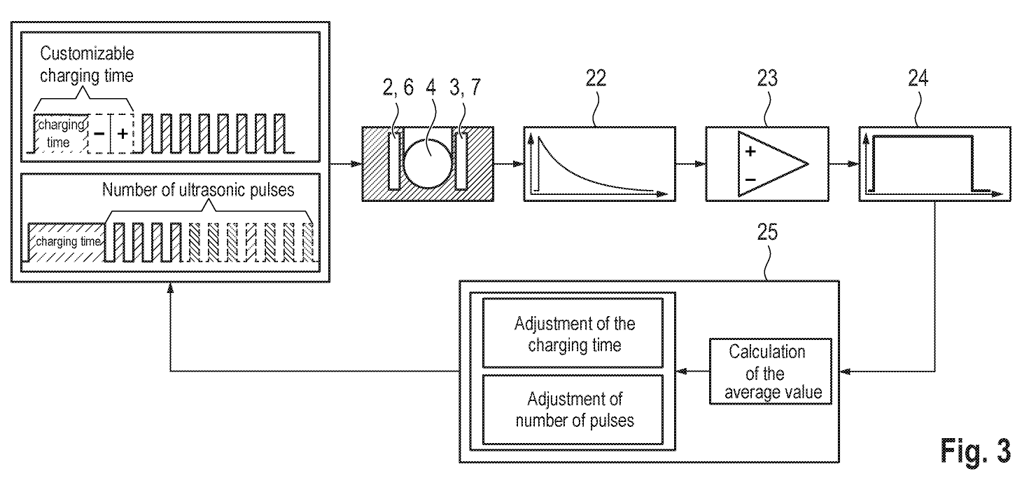

FIG. 3 illustrates how, according to an embodiment of the present invention, the charging time of the ultrasonic transmitter 2 and/or the number of ultrasonic pulses are adjusted to vary the amount of ultrasonic energy applied during the measurement. By combining adjustments to the charging time and the number of pulses, the energy content of the emitted pulses can be precisely controlled. An algorithm processes the steps shown in FIG. 3. The first window shows the parameters by which the ultrasonic energy can be varied. The charging time of the ultrasonic transmitter 2 and/or the number of ultrasonic pulses emitted by the ultrasonic transmitter 2 can be varied. After the ultrasonic pulses are emitted into the infusion tube 4, the ultrasonic receiver 3 receives ultrasonic signals 22. This is also possible with the configuration comprising the ultrasonic transmitter and receiver unit 6 as well as the ultrasonic reflector 7, wherein the ultrasonic pulses are guided from the ultrasonic transmitter and receiver unit 6 through the infusion tube 4 to the ultrasonic reflector 7, which in turn guides the pulses back through the infusion tube 4 to the ultrasonic transmitter and receiver unit 6. In this process, the individual ultrasonic pulses generate ultrasonic signals 22, which typically decrease in intensity exponentially over time. In the next step, the ultrasonic signals 22 are analyzed by a comparator 23, which is part of the control and evaluation unit 8. In this process, the time interval during which the ultrasonic signal 22 exceeds the currently set alarm threshold is determined (comparator signal 24). The average or another statistical measure is then calculated from several time intervals, based on which a control and evaluation unit 8 can adjust the number of ultrasonic pulses, depending on whether the average or another statistical measure lies within, above, or below a predefined tolerance range or value. In an alternative embodiment, the determination of the mean value or another statistical quantity and the resulting adjustment of the charging time of the ultrasonic transmitter or the number of ultrasonic pulses emitted per unit of time may be performed in a microcontroller unit 25, which is part of a control and evaluation unit 8.

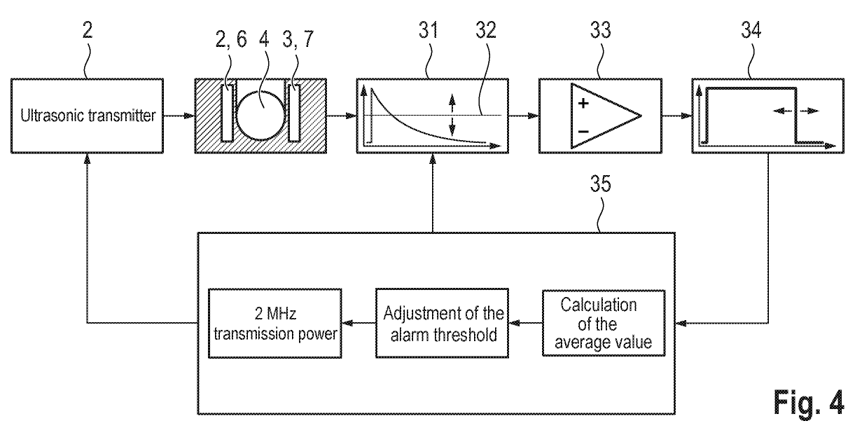

Alternatively, according to another advantageous embodiment of the underlying invention shown in FIG. 4, the level of the alarm threshold 32 can be adjusted. In this case, a control and evaluation unit 8 typically triggers an alarm as soon as the positive pulse width of the comparator signal 34 falls below a predetermined threshold value. The positive pulse width of the comparator signal 34 is determined by the time interval during which the ultrasonic signal 31 lies above the alarm threshold 32. The adjustment of the alarm threshold 32 proceeds as follows: The ultrasonic transmitter 2 transmits ultrasonic pulses through the infusion tube 4 to the ultrasonic receiver 3. This is also possible with a configuration comprising an ultrasonic transmitter and receiver unit 6 as well as an ultrasonic reflector 7, wherein the ultrasonic pulses are transmitted from the ultrasonic transmitter and receiver unit 6 through the infusion tube 4 to the ultrasonic reflector 7, which in turn transmits the pulses back through the infusion tube 4 to the ultrasonic transmitter and receiver unit 6. In this process, the individual ultrasonic pulses generate ultrasonic signals 31, which typically decrease in intensity exponentially over time. In the next step, the ultrasonic signals 31 are analyzed by a comparator 33, which is part of the control and evaluation unit 8. In this process, the time interval 34 is determined during which the ultrasonic signal 31 exceeds the currently set alarm threshold 32. The average or another statistical measure is then calculated from several time intervals, based on which the control and evaluation unit 8 can adjust the alarm threshold 32, depending on whether the average or another statistical measure lies within, above, or below a predefined tolerance range or value. In an alternative embodiment, the determination of the mean value or another statistical quantity and the resulting adjustment of the alarm threshold may be performed in a microcontroller unit 35, which is part of the control and evaluation unit 8.

In a particularly preferred embodiment, the two variants according to FIGS. 3 and 4 can be combined in a common control and evaluation unit 8 , and the described processes – adjustment of the alarm threshold and the transmission power – can run individually or together (simultaneously) as needed.

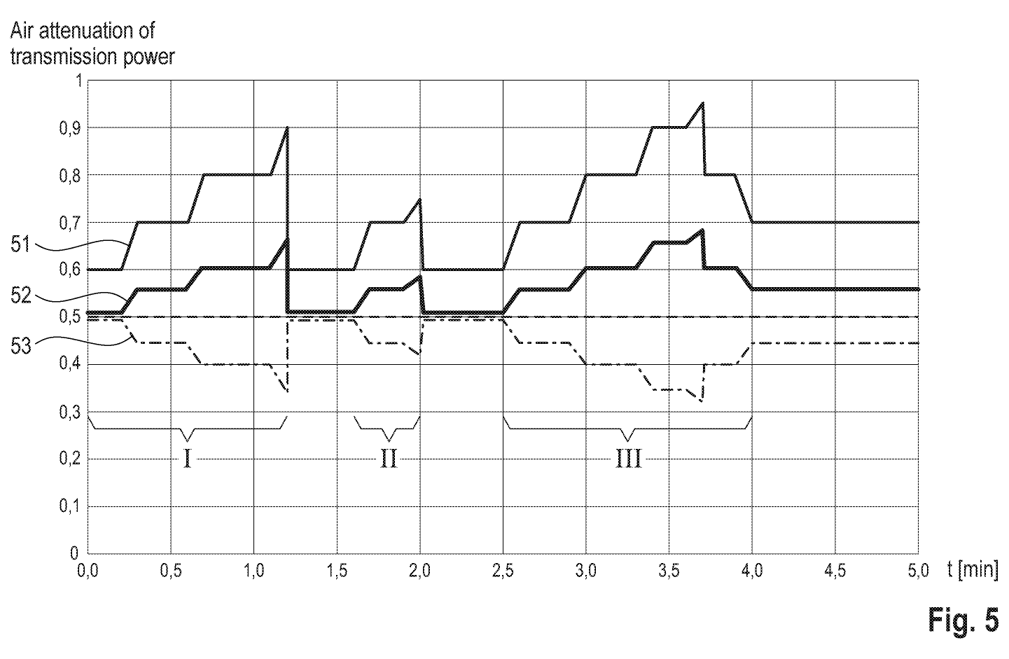

FIG. 5 shows an example of the attenuation of the ultrasonic signal 51 over time, which is processed by the control and evaluation unit 8 according to the invention. Based on the detected attenuation of the ultrasonic signal 51, the control and evaluation unit 8 regulates the alarm threshold 53 and the amount of coupled ultrasonic energy 52. In the present example, three stationary air bubbles initially accumulate one after another in the sensor region 5 of the infusion tube 4 (Section I in FIG. 5), as indicated by three increases in the attenuation of the ultrasonic signal 51 from an initial value of 0.6 to 0.9. After reaching this value, the attenuation of the ultrasonic signal 51 drops rapidly to a value of 0.6, indicating that the previously accumulated air bubbles have collectively left the sensor region 5. This is followed by two further increases in the attenuation of the ultrasonic signal, first from 0.6 to 0.7, followed shortly thereafter by an increase from 0.7 to 0.75 (Section II in FIG. 5). After reaching the last value, the attenuation of the ultrasonic signal 51 drops rapidly to 0.6. This curve represents the accumulation of two stationary air bubbles, the second of which is smaller than the first, whereby during the accumulation of the second air bubble, the air previously accumulated in the sensor area 5 is released and moves into the infusion tube 4. Subsequently (Section III in FIG. 5), there are three further increases in the attenuation of the ultrasonic signal 51, each by 0.1 from 0.6 to 0.9. This represents the accumulation of three air bubbles of equal size in the sensor area 5. After additional air accumulates in the sensor area 5, as indicated by the increase in the attenuation of the ultrasonic signal 51 from 0.9 to 0.95, the attenuation drops back to 0.8, corresponding to a partial release of the air previously accumulated in the sensor area 5. After a short time, the attenuation of the ultrasonic signal drops to a value of 0.7, which corresponds to a further removal of air from sensor area 5. Subsequently, the attenuation of the ultrasonic signal 51 remains constant. Compared to the lowest value of the attenuation of the ultrasonic signal 51 of 0.6, it can be deduced that a residual amount of air remains in sensor area 5. Proportional to the degree of attenuation of the ultrasonic signal 51, the control and evaluation unit 8 increases the amount of coupled ultrasonic energy 52 in order to maintain the sensitivity of the method or the gas sensor 1. Similarly, the alarm threshold 53 can be lowered accordingly to keep the system behavior for gas volume detection constant.

In an alternative example, a blood tube 10 (e.g., as part of an extracorporeal blood circuit) of a dialysis system is present instead of an infusion tube 4.

Overall, the described system enables adaptive detection capable of compensating for tolerances. The sensor can distinguish between stationary and moving bubbles and, via signal waveforms, also detects when individual gas bubbles leave the sensor area. Both their number and their approximate volume can be determined by analyzing the signal waveforms.

### LIST OF REFERENCE SYMBOLS

1 Gas sensor
2 Ultrasonic transmitter
3 Ultrasonic receiver
4 Infusion tube
5 Sensor region
6 Ultrasonic transmitter and receiver unit
7 Ultrasonic reflector
8 Control and evaluation unit
10 Blood tube
22 Ultrasonic signal
23, 33 Comparator
24, 34 Comparator signal
25, 35 Microcontroller
32, 53 Alarm threshold
51 Attenuation of the ultrasonic signal
52 Coupled ultrasonic energy

1. A method for detecting gas bubbles in an infusion tube through which an infusion medium flows or in a blood tube of a dialysis system through which blood flows, and in which an ultrasound-based gas sensor is used, the method comprising the steps of:
applying ultrasonic pulses to the infusion tube or blood tube by an ultrasonic transmitter;
detecting ultrasonic pulses propagated through the infusion tube or the blood tube by an ultrasonic receiver;
evaluating a sensor signal output by the ultrasonic receiver by a control and evaluation unit;
comparing the sensor signal or a signal derived the sensor signal with a threshold value to detect gas bubbles;
outputting an alarm signal when the sensor signal exceeds or falls below the threshold value; and
automatically adjusting or varying at least one parameter during the evaluating step.
the at least one parameter comprising:
a transmission energy introduced into the infusion tube or blood tube by means of the ultrasonic pulses, and
the threshold value.

2. The method according to claim 1, wherein the transmission energy is automatically adjusted during the evaluating step by controlling a number of successive ultrasonic pulses and/or by regulating an energy content of individual pulses.

3. The method according to claim 1, wherein the threshold value for triggering the alarm signal is dynamically adjusted as a function of the sensor signal over time.

4. The method according to claim 1, wherein:
the transmission energy is automatically adjusted during the evaluating step,
adjustment of the transmission energy is based on a predetermined target range for the sensor signal, and
the sensor signal is continuously compared with the predetermined target range.

5. The method according to claim 1, wherein automatic adjustment is performed by an algorithm implemented in the control and evaluation unit, which is operable to assign characteristic signal waveforms to physical phenomena in the infusion tube or blood tube.

6. The method according to claim 5, wherein the physical phenomena comprise stationary or moving gas bubbles.

7. The method according to claim 5, wherein the algorithm is based on a lookup table or on a machine learning model that was trained during operation or in advance.

8. The method according to claim 1, wherein, in addition to detection of gas bubbles, temporal movement of gas bubbles or removal of gas bubbles from a sensor area is detected, and a calculation of gas volume is performed based on signal waveforms.

9. The method according to claim 1, wherein the alarm signal is triggered only when the threshold value is exceeded for a predetermined minimum time or exceeds a certain slope of the sensor signal.

10. The method according to claim 1, wherein a plausibility check of sensor signals is performed to distinguish between gas bubble detection and signal changes due to tolerances or external interference.

11. The method according to claim 1, wherein evaluation of the sensor signal is performed continuously or at discrete intervals, depending on an operating mode of an associated infusion device or dialysis machine.

12. The method according to claim 11, wherein the operating mode is a preparation mode or therapy mode.

13. The method according to claim 1, wherein:
the control and evaluation unit is supplied with information regarding the infusion medium used, a flow rate, or a geometric position of the infusion tube or the blood tube, and
the information is incorporated into adjustment of the at least one parameter.

14. The method according to claim 1, wherein a volume of a gas bubble trapped in the infusion tube or blood tube is determined when the gas bubble detaches from the infusion tube or blood tube, by analyzing a signal rise in the sensor signal.

15. The method according to claim 14, further comprising the steps of automatically adjusting a transmission power and/or the threshold value, and incorporating the volume of the gas bubble into a total balance of detected gas bubble volumes.

16. A device for detecting gas bubbles in an infusion tube through which an infusion medium flows or in a blood tube through which blood flows in an infusion system, the device comprising:
an ultrasonic transmitter configured to apply ultrasonic pulses to the infusion tube or blood tube,
an ultrasonic receiver configured to detect ultrasonic pulses propagating through the infusion tube or blood tube,
a control and evaluation unit connected to the ultrasonic receiver and configured to evaluate a sensor signal generated by the ultrasonic receiver,
a comparison unit for evaluating the sensor signal or a signal derived from the sensor signal with respect to a threshold value for detecting gas bubbles,
an alarm unit for outputting an alarm signal when the sensor signal exceeds or falls below the threshold value, and
a control and evaluation unit configured to automatically adjust at least one of the following parameters during operation:
a transmission energy introduced by the ultrasonic pulses by varying a number of and/or an energy content of the ultrasonic pulses, and
the threshold value.

17. The device according to claim 16, wherein the control and evaluation unit is configured to regulate the transmission energy based on a predetermined target range for the sensor signal.

18. The device according to claim 17, wherein the control and evaluation unit is configured to regulate the transmission energy by comparing a current signal magnitude with the predetermined target range and adjusting a number and/or energy content of the ultrasonic pulses accordingly.

19. The device according to claim 17, wherein the control and evaluation unit implements an algorithm configured to assign different signal waveforms to characteristic occurrences in the infusion tube or blood tube.

20. The device according to claim 19, wherein the characteristic occurrences comprises stationary or moving gas bubbles.
